(19) United States
(12) Patent Application Publication
(10) Pub. No.: US 2026/0231980 A1
(43) Pub. Date: Aug. 13, 2026

(54) Method of producing a high-protein food product from brewery's spent grain

(71) Applicant: Luonnonvarakeskus, Helsinki (FI)

(72) Inventors: Henri Vanhanen, Helsinki (FI); Seija Repo, Helsinki (FI); Marta Cortina Escribano, Helsinki (FI); Juho Haveri-Heikkilä, Helsinki (FI); Jarkko Hellström, Helsinki (FI); Pertti Marnila, Helsinki (FI); Juha-Matti Pihlava, Helsinki (FI); Petri Kilpeläinen, Helsinki (FI)

(21) Appl. No.: 19/155,263

(22) PCT Filed: Feb. 9, 2024
(86) PCT No.: PCT/FI2024/050052

§ 371 Date: Aug. 11, 2025

(30) Foreign Application Priority Data
Feb. 10, 2023 (FI) 20235139

(51) Int. Cl. A23J 3/22; A01G 18/20; A01G 18/50; A23J 1/12; A23J 3/14; A23J 3/26; A23K 10/38

(52) U.S. Cl. CPC A23J 3/227; A01G 18/20; A01G 18/50; A23J 1/12; A23J 3/14; A23J 3/26; A23K 10/38

(57) ABSTRACT

The present invention provides a method of producing a grain derived high-protein food product, comprising the steps of: a) providing a biomass substrate, wherein said biomass substrate comprises at least one type of grain, preferably brewer's spent grain; b) subjecting said biomass substrate to a heat treatment in order to sterilize the substrate; c) inoculating the sterilized substrate obtained from step b) with a fungal strain; and preferably forming the substrate into a predetermined shape suitable for a solid flatbed culture; and d) incubating the inoculated biomass substrate obtained in step c) at 15-30° C. in an aerobic environment to grow a network of fungal mycelium into the biomass substrate in order to improve the structure and flavor of the product. The present invention also provides a novel Pleurotus ostreatus strain.

### FIELD

The present invention relates to field of production of plant-protein based food products and, in particular, to utilization of a byproduct of the brewing industry, namely brewer's spent grain, and more specifically to a method for growing fungal mycelium and preparing a high-protein food product for human consumption.

### BACKGROUND

Plant-protein based food is gaining acceptance due to health concerns and environmental issues related with the production of meat. For example, with the production of beef meat large agricultural areas are involved in order to provide sufficient feed for the animals. However, within the food sector there are still relatively few meat substitutes which aim to provide the proteins which are recommended for daily intake. Another problem in the field is that current meat substitutes do not have an appealing texture and taste. Attempts have been made to solve the problems by letting mushroom mycelium grow into suitable edible substrates.

EP2835058 discloses a method for the production of a meat substitute composition based on solid state fermentation, wherein the method comprises the steps of: (i) providing a substrate comprising a cereal and a plant derived product or a fish derived product and having a moisture content of above 50% (w/w); (ii) introducing to said substrate an edible mushroom mycelium; and (iii) allowing said mycelium to grow in said substrate for a period which is sufficient to saturate the substrate with mycelium to provide the meat substitute composition.

WO2020232347 discloses a method for a preparation of a protein food product for human or animal consumption, comprising the steps of providing a sterilized substrate comprising a grain and a plant protein concentrate or isolate, wherein the substrate is at least 50% protein isolate or concentrate by dry weight, and inoculating the sterilized substrate with a filamentous fungal culture in solid state fermentation conditions; and culturing the filamentous fungal culture and the sterilized substrate, wherein the filamentous fungal culture grows hyphae and forms a mycelial network to form protein food product, wherein the protein food product has increased desirable flavors and/or reduced undesirable aromas and/or flavors compared to a non-myceliated control substrate.

In view of the above, there is still a need in the art for the provision of an improved meat or sea food substitute having a texture which is meat-like or sea food-like, comprises essential amino acids and provides a desired taste.

### SUMMARY OF THE INVENTION

The invention is defined by the features of the independent claims. Some specific embodiments are defined in the dependent claims.

According to a first aspect of the present invention, there is provided a method of producing a food product, comprising the steps of:
a) providing a biomass substrate, wherein said biomass substrate comprises at least one type of grain, preferably brewer's spent grain;
b) subjecting said biomass substrate to a heat treatment in order to sterilize the substrate;
c) inoculating the sterilized substrate obtained from step b) with a fungal strain; and preferably forming the substrate into a predetermined shape suitable for a solid flatbed culture; and
d) incubating the inoculated biomass substrate obtained in step c) at 15-30° C. in an aerobic environment to grow a network of fungal mycelium into the biomass substrate to produce the food product.

According to a second aspect of the present invention, there is provided a food product obtained by the method described above.

According to a third aspect of the present invention, there is provided a grain derived high-protein food product comprising grain, preferably brewer's spent grain, and a network of fungal mycelium of a fungal strain in the product

According to a fourth aspect of the present invention, there is provided a Pleurotus ostreatus strain deposited at the Westerdijk Fungal Biodiversity Institute Culture Collection with accession number CBS 149650, wherein incubating said strain in a biomass substrate comprising brewer's spent grain provides a network of fungal mycelium into the biomass substrate and an improved structure for said substrate.

### BRIEF DESCRIPTION OF THE DRAWINGS

FIG. 1. Alternative process workflows for producing a high-protein food product using brewer's spent grain. (A) Basic method steps. B1=Brewers's spent grain; B2=Ingredients, such as those listed in Example 1 below; B3=Substrate; B4=Sterilized substrate; B5=Inoculum of the fungal strain; B6=Product. (B) Basic method steps with a UV treatment. B1=Brewers's spent grain; B2=Ingredients; B3=Substrate; B4=Sterilized substrate; B5=Inoculum of the fungal strain; B6=Product; B7=D-vitamin enriched product. (C) Basic method steps with an extrusion step. B1=Brewers's spent grain; B2=Ingredients; B3=Substrate; B4=Sterilized substrate; B5=Inoculum of the fungal strain; B6=Product mass; B7=Product.

### EMBODIMENTS

In the present context, the term “Brewer's Spent Grain” or “Brewery Spent Grains”, BSG, is defined as a byproduct produced in the process of beer production by the brewing industry.

More specifically, BSG can be defined as the leftover barley, malt, oats, rye, rice, wheat or mixtures thereof remaining after the mash mixture has been extracted from most of the sugars and other carbohydrates during brewing. BSG is a lignocellulosic material containing about 70% fiber (cellulose, non-cellulosic polysaccharides and lignin) and about 20% protein. In addition to its high fiber and protein content, BSG contains beneficial polyphenolic antioxidants, all of which contribute to the positive nutritional value of BSG. BSG is rich in carbohydrates and proteins but the main use to date for the utilization of this product has been as animal feed.

BSG is distinct and separate from brewery “sludge”. In practice, brewery sludge is easily distinguishable from BSG by those skilled in the brewery arts.

Further descriptions of the uses and compositions of BSG can be found from review articles by Xiros and Christakopoulos, 2012; Mussatto et al., 2004; and Lynch et al., 2016.

In the present context, the term “grain” is referring to any known cultivated grain species, preferably said grain is selected from the group consisting of: barley (Hordeum vulgare), oat (Avena sativa), rye (Secale cereale), corn (Zea mays), wheat (Triticum spp.), bulgur, farro, spelt, emmer, einkorn, freekeh (Triticum spp.), millet (Panicum miliaceum, Pennisetum glaucum, Setaria italica), Sorghum spp., amaranth (Amaranthus cruentus), buckwheat (Fagopyrum esculentum), and quinoa (Chenopodium quinoa). More preferably, said grain is barley, oat or rye. The grains of the present disclosure also include industrial side stream grains such as Brewer's Spent Grain.

The present invention is based on a method of culturing filamentous fungus in a solid culture in an aerobic environment using a sterilized biomass substrate that contains at least one type of grain, preferably Brewer's Spent Grain, BSG, wherein a network of fungal mycelium grows into the biomass substrate (i.e. said mycelium grows into said substrate for a period of time which is sufficient to provide the meat/sea food substitute properties for the food product of the present disclosure). In this method, a composition comprising a protein food product which is similar in structure, taste and nutritional value to meat or sea food can be provided. The inventors found that the treatment with a fungal strain can also alter the taste, flavor or aroma of the biomass substrate to provide umami flavor to the substrate material. Further, the treatment also provides a texture after cooking, which is similar to that of cooked texture of actual meat or sea food. It is thus the present inventors' finding that the culturing of biomass comprising BSG with any fungal strain of the present disclosure leads to a myceliation process providing texture which is, when cooked, surprisingly similar to cooked ground meat or sea food. The myceliation causes growth of fungal hyphae to form a mycelial network into the substrate to allow the composition to be more cohesive compared to an unmyceliated composition.

Accordingly, in an embodiment, the present invention provides a method of producing a food product, the method comprising the steps of:
a) providing a biomass substrate, wherein said biomass substrate comprises at least one type of grain, preferably brewer's spent grain;
b) subjecting said biomass substrate to a heat treatment in order to sterilize the substrate;
c) inoculating the sterilized substrate obtained from step b) with a fungal strain; and preferably forming the substrate into a predetermined shape suitable for a solid flatbed culture; and
d) incubating the inoculated biomass substrate obtained in step c), preferably at least 7 days, more preferably 7-28 days, at 15-30° C. in an aerobic environment to grow a network of fungal mycelium into the biomass substrate in order to improve the structure and flavor of the food product produced.

In a preferred embodiment, grain such as brewer's spent grain accounts for at least 10% (w/w), 20% (w/w), 30% (w/w), 40% (w/w), 50% (w/w), 60% (w/w), 70% (w/w), or 80% (w/w) of the biomass substrate (dry weight), which is subjected to the heat treatment in step b). In a more preferred embodiment, grain such as brewer's spent grain accounts for 10%-80% or 40%-80% (w/w) of the biomass substrate (dry weight).

In a preferred embodiment, said fungal strain is selected from the strains of a fungal genus selected from the group consisting of Pleurotus spp., Hericium spp., Inonotus spp., and Ganoderma spp.

In a more preferred embodiment, said fungal strain is selected from the strains of fungal species of the group consisting of Pleurotus ostreatus, Hericium erinaceus, Hericium coralloides, Inonotus obliquus, and Ganoderma sp. (known also as G. lucidum). In a more preferred embodiment, said fungal strain is a Pleurotus ostreatus strain deposited at the Westerdijk Fungal Biodiversity Institute with accession number CBS 149650. In another preferred embodiment, said fungal strain is a Hericium erinaceus or Hericium coralloides strain providing sea food-like properties to the food product.

In a preferred embodiment, the heat treatment in step b) is performed at 100-135° C. for 20-300 min. A skilled person can easily modify the time and temperature depending on the volume of the biomass, the device, and receptacles used for the treatment.

In another preferred embodiment, the method comprises a preceding step of milling, grinding or extrusion of the wet or dried brewer's spent grain (preferably frozen brewer's spent grain) in order to adjust (decrease) the particle size of the substrate, preferably before mixing said brewer's spent grain with other ingredients of the biomass substrate. Crushing the brewer's spent grain improves the structure of the substrate: the grain size is reduced and the texture of the biomass is more solid.

In an embodiment, inoculation of the sterilized substrate by the fungal inoculum in step c) may be carried out by any methods known in the art, including, without limitation, injection into the substrate, and spraying or pipetting inoculum onto the surface of the substrate. In a preferred embodiment, the present method comprises preceding steps of culturing said fungal strain in a liquid culture, preferably in a potato dextrose broth, and preparing an aliquot or concentrate of said liquid culture for inoculation step c).

In an embodiment, the incubating step d) may be performed by methods known in the art and may be carried out in a sealed culturing box and/or in a culturing bag preferably forming the substrate into a predetermined shape suitable for a solid flatbed culture to permit development of hyphae and a mycelial network while preventing contaminations. In one embodiment, this process consists of depositing a solid biomass substrate, as disclosed herein, on flatbeds after seeding it with a fungal strain; the substrate is then left in a temperature-controlled room for several days at 15-30° C. in an aerobic environment to grow a network of fungal mycelium into the biomass substrate.

Sterilization of the biomass substrate preceding the culturing step may be performed as is known in the art. Substrate may be sterilized in a container. In an embodiment, the container is the bag or box which is also used for the subsequent culturing step. In another embodiment, the sterilized biomass substrate is packed in culture boxes/bags for performing step d), said boxes/bags preferably comprising openings or pores providing said aerobic environment.

In the present invention, the food product obtained in step d) is preferably subjected to UV irradiation during processing (preferably during the incubation step or at the end of the incubation step), being irradiated with UV light for a time sufficient to enhance the vitamin D content thereof. By utilizing UV irradiation, the food product has a substantially increased level of vitamin D. In an embodiment, the biomass is irradiated with UV radiation during the growing process, specifically Ultraviolet-B (UV-B), a section of the UV spectrum, with wavelengths between about 280 and 320 nm, or Ultraviolet-C (UV-C), with wavelengths between about 200 and 280 nm. It is believed that the additional vitamin D is obtained through the conversion of ergosterol due to the UV irradiation. The treatment time will normally be between 5 minutes and 12 hours, more preferable, 2 hours, wherein the cultured biomass is preferably subjected to short-term (such as 1 s) irradiance at regular intervals.

In addition to BSG, the biomass substrate may additionally comprise one or more of vegetables materials. The vegetable materials or substances can be obtained from any of several vegetable sources and can include one or more of the vegetables freshly grated or sliced, as extracts, or dried or partially dried form, e.g., powders. Vegetables suitable for the present invention include any prepared from a vegetarian source such as carrot, spinach, beans kale, beet, celery, broccoli, cauliflower, watercress, Chinese cabbage, chard, beet greens, chicory, leaf lettuce, parsley, romaine lettuce, collard greens, turnip greens, mustard greens, sunflower, bell pepper, arugula, pumpkin, brussel sprout, cabbage, turnip, potato, sweet potato, or a combination thereof. In a preferred embodiment, said biomass substrate comprises carrot and/or beet, preferably grated carrot and/or beet. In another preferred embodiment, said biomass substrate comprises potato powder or potato protein powder. In the experiments performed, it was shown that particularly beetroot and/or carrot deepen the taste of the final product and also significantly increase the growth of the mycelium into the structure.

In another preferred embodiment, the biomass substrate may also additionally comprise added calcium carbonate, CaCO₃.

In another preferred embodiment, said biomass substrate comprises oats and/or broad bean (Vicia faba) powder. Broad bean powder adds more tryptophan to the final product, thus a desired amino acid composition is achieved for the product.

In another preferred embodiment, said biomass substrate is a mix of brewer's spent grain, grated carrot, grated beet, potato powder, added calcium carbonate, oats, and broad bean (Vicia faba) powder.

In another preferred embodiment, said biomass substrate is a mix of brewer's spent grain, and one or more ingredients selected from the group consisting of: grated carrot, grated beet, potato powder, potato protein powder, added calcium carbonate, oats, and hemp protein powder.

In order to improve nutritional values as well as taste of the final food product, the present method may comprise a further step of adding a mushroom extract to the product obtained from step d), wherein said mushroom extract is preferably prepared by subjecting fruiting bodies (sporocarps) of the fungal strain to water or alcohol extraction.

Further to above embodiments, the present invention is directed to a brewer's spent grain derived high-protein food product comprising brewer's spent grain and a network of fungal mycelium in the product. In a preferred embodiment, the food product comprises one or several of the following: grated carrot, grated beet, potato powder, potato protein, seaweed, hemp powder, hemp protein, added calcium carbonate, oats, and broad bean (Vicia faba) powder.

In another preferred embodiment, said food product is a ready-to-cook food product.

In another preferred embodiment, said food product is a meat substitute or a sea food substitute.

In another preferred embodiment, said food product is flavored with meat flavoring and/or by meat addition in order to further improve flavor of the food product.

In another preferred embodiment, said food product is flavored with sea food flavoring and/or by sea food addition in order to improve flavor of the food product.

In another preferred embodiment, said food product is flavored with spruce sprouts flavoring of Abies spp, Pinus spp. or any Pinaceae family species and/or by addition of said sprouts in order to improve flavor of the food product.

The present invention is also directed to a Pleurotus ostreatus strain deposited to the culture collection at the Westerdijk Fungal Biodiversity Institute with accession number CBS 149650, wherein incubating said strain in a biomass substrate comprising brewer's spent grain provides a network of Pleurotus ostreatus mycelium into the biomass substrate and an improved meat-like structure for said substrate. Accordingly, the applicant states that the Pleurotus ostreatus strain deposited to the culture collection at the Westerdijk Fungal Biodiversity Institute (address: Uppsalalaan 8, 3584 CT, the Netherlands) with accession number CBS 149650 was deposited by the applicant Luonnonvarakeskus (LUKE) on Dec. 6, 2022, according to the Regulations of the Budapest Treaty.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, i.e. a singular form, throughout this document does not exclude a plurality.

### EXPERIMENTAL SECTION

### Example 1. Production of a Brewer's Spent Grain Derived High-Protein Food Product

A strain of Pleurotus ostreatus was first cultivated in a liquid culture composed of potato dextrose broth. The liquid culture was kept in a rotary shaker (90 rpm) in dark conditions at 22° C. for 7 days. Mycelial pellets were formed in the liquid culture. The substrate media was prepared by mixing the ingredients as follow: dry grated beet (19.5 g/kg), potato powder (48.8 g/kg), calcium carbonate (26 g/kg) and broad bean powder (97.7 g/kg). Hemp protein powder (97.7 g/kg) or potato protein powder (97.7 g/kg) can be used as an alternative to broad bean powder.

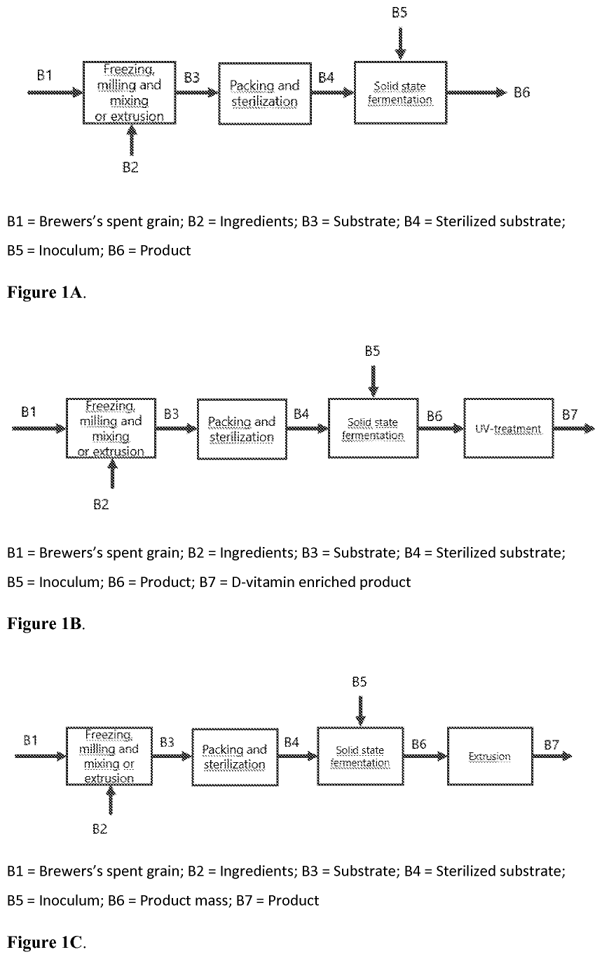

Spent brewery grains, BSG, (664.5 g/kg) were added and mixed with the above-mentioned ingredients. The substrate media was packed in a polypropylene box and sterilized for 90 minutes at 125° C. and a pressure of 1.2 bar. The sterilized substrate was kept in sterile conditions until cooling down at room temperature. The sterilized substrate was inoculated with the liquid culture (25 mL/kg) containing Pleurotus ostreatus mycelia. The inoculated substrate was then incubated in dark conditions at 22° C. for 10 days to produce the final product. General process workflow options are shown in FIG. 1.

### Example 2. Measurement of Major Nutrients of the High-Protein Food Product

The major nutrients were measured and calculated on dry basis. The results are shown in Table 1 below. Conclusions were the following: i) the product of the present disclosure has a high fiber content (carbohydrates 59 g/100 g); ii) the product of the present disclosure has a high betaglucan content (4.6-6 g/100 g); iii) the protein content of the product (28 g/100 g) is higher than regular plant-based ready-to-cook products currently on market (19-21 g/100 g); and iv) the fat content of the product (3.6 g/100 g) is essentially lower than in many ready-to-cook meat products currently on market (40-50 g/100 g).

TABLE 1
Major nutrients measured in the high-protein
food product prepared in Example 1.

| | | | | Moisture |
|---|---|---|---|---|
| Carbohydrates | Protein | Fat | Ash | content |
| (g/100 g) | (g/100 g) | (g/100 g) | (g/100 g) | (%) |
| 59 (57.8-61.0) | 28 (27.3- | 3.6 (3.0- | 9.3 (8.2- | 55-70 |
| From which | 29.2) | 4.4) | 11.5) | |
| betaglucan 4.6-6.0 | | | | |

### Example 3. Suitability of Fungal Species or Strain of Fungal Species for Production of the High-Protein Food Product

Several strains of fungal species were tested for their suitability for growing the high-protein product as described in Example 1. In Table 2 below, a level of good features of fungal species are marked with (+, ++, +++) and bad features ruling out the possibility for production by (−). Successful growth in the substrate, colonization time and mycelial density were assigned as the primary parameters to estimate the capability and feasibility to produce the product described. No fruiting body production on the substrate indicates that the species does not produce fruiting bodies during the incubation and production cycle. Fruiting bodies production would deter the quality of the product.

TABLE 2
Suitability of fungal species for production
of the high-protein food product.

| | Succesful | Coloni- | | No fruiting body |
|---|---|---|---|---|
| | growth in the | zation | Mycelial | production on |
| Fungal species | substrate | time | density | the substrate |
| Ganoderma sp. | ++ | + | ++ | +++ |
| (lucidum) | | | | |
| Grifola | + | + | − | +++ |
| frondosa | | | | |
| Hericium | + | + | + | +++ |
| coralloides | | | | |
| Laetiporus | + | − | − | +++ |
| sulphureus | | | | |
| Polyporus | ++ | +++ | + | − |
| squamosus | | | | |
| Pleurotus | ++ | +++ | + | − |
| pulmonarius | | | | |
| Kuehneromyces | ++ | + | − | ++ |
| mutabilis | | | | |
| Pleurotus | +++ | +++ | +++ | +++ |
| ostreatus | | | | |
| (Saimaa) (CBS | | | | |
| 149650) | | | | |
| Inonotus | +++ | ++ | +++ | +++ |
| obliquus | | | | |
| Hericium | + | + | + | +++ |
| erinaceus | | | | |

### Example 4. Production of a Cereal Grain Derived High-Protein Product

A strain of Pleurotus ostreatus (CBS 149650) was cultivated in potato dextrose agar at 22° C. for 5 days. Five 1 cm² pieces of mycelium colonized agar were transferred to a liquid culture composed of potato dextrose broth, which was previously sterilized for 60 minutes at 125° C. and a pressure of 1.2 bar. The liquid culture was kept in a rotary shaker (90 rpm) in dark conditions at 22° C. for 7 days. Mycelial pellets were formed in the liquid culture. The substrate media was prepared by mixing the ingredients as follow: dry grated beet (19.5 g/kg), potato powder (48.8 g/kg), calcium carbonate (26 g/kg) and broad bean powder (97.7 g/kg). Hemp protein powder (97.7 g/kg) or potato protein powder (97.7 g/kg) can be used as an alternative to broad bean powder.

Oat grains (664.5 g/kg) were boiled for 45 minutes and mixed with the above-mentioned ingredients. Barley grains (664.5 g/kg) or rye grains (664.5 g/kg) can be used as an alternative grain substrate. The substrate media was packed in a polypropylene bag and sterilized for 90 minutes at 125° C. and a pressure of 1.2 bar. The sterilized substrate was kept in sterile conditions until cooling down at room temperature. The sterilized substrate was inoculated with the liquid culture (25 mL/kg) containing Pleurotus ostreatus mycelia. The inoculated substrate was then incubated in dark conditions at 22° C. for 10 days to produce the food product.

### CITATION LIST

### Patent Literature

EP2835058
WO2020232347

### Non-Patent Literature

Lynch K M, Steffen E J, and Arendt E K, Brewers' spent grain: a review with an emphasis on food and health, J. Inst. Brew. 2016; 122:553-568.
Mussatto S I, Dragone G, and Roberto I C, Brewers' spent grain: generation, characteristics and potential applications, Journal of Cereal Science 43 (2006) 1-14.
Xiros C and Christakopoulos P, Biotechnological Potential of Brewers Spent Grain and its Recent Applications, Waste Biomass Valor (2012) 3:213-232.

1. A method of producing a food product, comprising the steps of:
a) providing a biomass substrate, wherein said biomass substrate comprises at least one type of grain;
b) subjecting said biomass substrate to a heat treatment in order to sterilize the substrate;
c) inoculating the sterilized substrate obtained from said subjecting step b) with a fungal strain; and forming the substrate into a predetermined shape suitable for a solid flatbed culture; and
d) incubating the inoculated biomass substrate obtained in said inoculating step c) at 15-30° C. in an aerobic environment to grow a network of fungal mycelium into the biomass substrate to produce the food product.

2. The method according to claim 1, wherein said fungal strain does not produce fruiting bodies or produces only a few fruiting bodies during the incubating step d).

3. The method according to claim 1, wherein said fungal strain is selected from the strains of a fungal genus selected from the group consisting of Pleurotus spp., Hericium spp., Inonotus spp., and Ganoderma spp.

4. The method according to claim 1, wherein said fungal strain is selected from a group consisting of strains of fungal species Pleurotus ostreatus, Hericium erinaceus, Hericium coralloides, Inonotus obliquus, and Ganoderma sp.

5. The method according to claim 4, wherein said fungal strain is a strain of fungal species Pleurotus ostreatus.

6. The method according to claim 5, wherein said fungal strain is the Pleurotus ostreatus strain deposited at the Westerdijk Fungal Biodiversity Institute with accession number CBS 149650.

7. The method according to claim 1 comprising preceding steps of culturing said fungal strain in a liquid culture and preparing an aliquot or concentrate of said liquid culture for the inoculation step c).

8. The method according to claim 1 comprising a preceding step of milling, grinding or extrusion of frozen, wet or dried brewer's spent grain in order to adjust the particle size of the brewer's spent grain before mixing said brewer's spent grain with other ingredients of the biomass substrate.

9. (canceled)

10. The method according to claim 1, wherein said biomass substrate is packed in culturing bags and/or boxes, said bags and/or boxes preferably comprising openings or pores providing said aerobic environment.

11. (canceled)

12. The method according to claim 1, wherein said food product is a meat or sea food substitute.

13. (canceled)

14. The method according to claim 1, wherein said biomass substrate comprises oats, broad bean (Vicia faba), hemp protein and/or potato protein powder.

15. The method according to claim 1, wherein said biomass substrate is a mix of brewer's spent grain, and one or more of the following: grated carrot, grated beet, seaweed, potato powder, potato protein, hemp powder, hemp protein, added calcium carbonate, oats, and broad bean (Vicia faba) powder.

16. The method according to claim 1, wherein said grain is selected from the group consisting of: barley (Hordeum vulgare), oat (Avena sativa), rye (Secale cereale), corn (Zea mays), wheat (Triticum spp.), bulgur, farro, spelt, emmer, einkorn, freekeh (Triticum spp.), millet (Panicum miliaceum, Pennisetum glaucum, Setaria italica), Sorghum spp., amaranth (Amaranthus cruentus), buckwheat (Fagopyrum esculentum), and quinoa (Chenopodium quinoa).

17. (canceled)

18. The method according to claim 1, wherein said grain is brewer's spent grain.

19. (canceled)

20. A food product comprising grain and a network of fungal mycelium of a fungal strain in the product.

21. The food product according to claim 20, wherein said grain accounts for 10%-80% (w/w) of the ingredients used for the preparation of the product.

22. The food product according to claim 20, wherein said fungal strain is selected from a group consisting of strains of fungal species Pleurotus ostreatus, Hericium erinaceus, Hericium coralloides, Inonotus obliquus, and Ganoderma sp.

23. The food product according to claim 22, wherein said fungal strain is the Pleurotus ostreatus strain deposited at the Westerdijk Fungal Biodiversity Institute with accession number CBS 149650.

24. The food product according to claim 20 comprising milled, extruded, or grinded brewer's spent grain.

25-30. (canceled)

31. Pleurotus ostreatus strain deposited at the Westerdijk Fungal Biodiversity Institute with accession number CBS 149650.

32-34. (canceled)
